(19) United States
(12) Patent Application Publication
(10) Pub. No.: US 2026/0007792 A1
(43) Pub. Date: Jan. 8, 2026

(54) COMPOSITIONS FOR DENATURATION OF RESPIRATORY ALLERGENS

(71) Applicant: IFL PHARMA ILAC SANAYI VE TICARET ANONIM SIRKETI, Golbasi, Ankara (TR)

(72) Inventors: Naz Dicle BALKAN GURSES, Golbasi, Ankara (TR); Mahir TULUNAY, Golbasi, Ankara (TR)

(21) Appl. No.: 18/881,557

(22) PCT Filed: Sep. 16, 2022
(86) PCT No.: PCT/TR2022/050994

§ 371 Date: Jan. 6, 2025

(30) Foreign Application Priority Data
Aug. 16, 2022 (TR) 2022/012958

(51) Int. Cl. A61L 2/20; A61L 101/44; A61L 101/46; A61L 101/50

(52) U.S. Cl. CPC A61L 2/20; A61L 2101/44; A61L 2101/46; A61L 2101/50

(57) ABSTRACT

Disclosed are compositions developed for the denaturation of respiratory allergens, such as mite, pollen, feline, canine, mold and fungal allergens. In particular, disclosed are water based compositions of vegetable origin, which prevent allergic reactions in the body by denaturing respiratory allergens before they enter the body through the respiratory tract, while they are still in the environment. Different versions of compositions are disclosed containing at least one acid, at least one herbal essential oil, an anionic surfactant or cyclodextrin, as well as water.

### SUBJECT OF THE INVENTION AND TECHNICAL FIELD

The invention relates to compositions developed for the denaturation of respiratory allergens, such as mite, pollen, feline, canine, mold and fungal allergens. The invention in particular relates to water based compositions of vegetable origin, which prevent allergic reactions in the body by denaturing respiratory allergens before they enter the body through the respiratory tract, while they are still in the environment. The invention includes versions of compositions containing an anionic surfactant or cyclodextrin as well as at least one acid, at least one herbal essential oil, and water.

### STATE OF THE ART

Today, the number of allergies and patients with allergy is increasing rapidly worldwide. Under normal conditions, the immune system is responsible for detecting foreign proteins, such as parasites, bacteria and viruses and rendering them harmless. It achieves this by producing antibodies against the foreign substance. Antibodies activate the resistant cells by providing a connection between the foreign protein and some resistant cells of the body and enable the foreign substances to be eliminated from the body. The immune system, which protects the body, also overreacts to certain substances which are not harmful in some people. These reactions are called “hypersensitivity” or “allergy”. Substances which cause an allergic reaction are called “allergens”. These substances may be taken by inhalation, skin contact, or orally in the form of food.

The allergens entering through the respiratory tract, such as mite, pollen, feline, canine, mold and fungal allergens are defined as respiratory allergens. DER P1, DER P2 and DER F1, DER F2 are some of the mite allergens in the state of the art. Fel d 1, Fel d 2 and Can f 1, Can f 2 are some of the pet allergens in the state of the art. These allergens are of feline and canine origin. rAlt a 1, rAsp f1, rCla h 8, Aspergillus fumigatus, Penicillium notatum are some of the mold and fungal allergens. Betula pendula (Common birch), Quercus L. (Oak tree), Arnica chamissonis (Grass), Populus (Poplar), Salix Salicaceae are some of the pollen allergens.

In patients with allergy, when the respiratory allergens enter the body through the respiratory tract, they are captured by the immunoglobulin E antibody in the mast cells and cause histamine release and allergic reactions due to this histamine release. During the binding of the allergens to the immunoglobulin E antibody, there is a mechanical lock and key relationship as in the enzyme-substrate interaction.

Allergic people generally apply the following methods to prevent allergic symptoms.
Antihistamines: Allergic people may start taking such drugs a few weeks before the start of the allergy season by the guidance of their doctor.
Immunotherapy tablets, or drugs used to desensitize the body to the allergens.
Nasal sprays designed to relieve itching or congestion: These include decongestants, which are a short-term solution to swelling.
Nasal corticosteroid sprays effective in reducing inflammation and the associated symptoms in the nasal passages.
Brooming and dusting processes.

Drugs such as decongestants, antihistamines, corticosteroids mentioned here are short-term solutions and have side effects on the patient. Brooming and dusting processes, or storing various of textile products such as mattress covers-pillow cases in deep freezers are processes requiring both time and effort. On the other hand, in the state of the art, alcohol-based disinfectants are one of the ways applied by the people exposed to allergens. These disinfectants cannot be used against all types of allergens and pose a safety risk due to glowing/flammability. In the state of the art, some patent studies for the destruction of allergens are as follows:

US patent no. US2003086991A1 relates to a method for inactivating Der P and Der F allergens. Said method uses essential oils. The essential oil is delivered to the area to be treated by heating, or by means of an ultrasonic jet nebulizer. Said essential oil is stated to be selected from cajeput oil, or oils containing at least one terpene hydrocarbon.

US patent no. US2003118670A1 has claimed compositions and methods for preventing or alleviating allergic reactions. These compositions, especially composed of acidic salt solutions of aluminum, calcium or magnesium, change the physical and/or chemical structure of the allergen proteins and reduce the degree of allergenicity.

Japanese patent no. JP2007326992A is a study to deactivate the pollen and mite allergens. Pollen allergen deactivator contains an extract from the leaf of the star fruit as an active ingredient. Mite allergen deactivator contains an extract obtained from the leaf of star fruit and/or brown rice.

US patent no. US2007134350A1 describes a method developed to deactivate the mite allergens. In said method, a mixture of oils such as citrus oil, peppermint oil, rose oil, jasmine oil, frankincense, bergamot oil and lemon oil is used.

Japanese patent no. JP2003334237A relates to an allergen-removing composition which is claimed to be able to effectively remove allergens, such as acarids, organisms, corpses, feces, etc. using a simple cleaning process. In this study, upon formation of an azeotropic mixture between the allergen and the solution by spraying a solution prepared to the environment/onto the surface and evaporation of the aqueous phase, the solid form consisting of the remaining inorganic compounds and allergens is removed from the environment by using a cleaning means such as a cleaning cloth, vacuum cleaner, after being dried completely.

US patent no. US2009275493A1 describes an application in which the allergens are hydrolyzed by using detergents and enzymes found in detergents, such as protease.

The invention of the Turkish Patent No. 2016/06496 to the inventor is related to a vacuum cleaner and chemical composition, which denature mites and mite proteins. Here is a study including only mite allergen proteins. The chemical in this invention is applied only to the vacuum cleaner.

Accordingly, in the state of the art, it is seen that studies on allergens are mostly focused on the detection of allergens, reducing the effects of the allergens, treating and curing the allergic disorders, but the studies on the denaturation of the allergens are few in number and open to development. There are very few studies on the denaturation of the allergens which may be exposed through the respiratory tract. The type of the allergens for which the current studies affect has been seen to be limited. Accordingly, there is a need for products easy to apply, which comprise a water based drug product of vegetable origin, may be an alternative to the solutions to which the patients with allergy apply in the state of the art and do not cause any problem including spending time and expending energy with the brooming and dusting processes, side effects caused by the drugs, risks for the alcohol based disinfectants to glow/burn.

### Technical Problems to be Solved by the Invention

The object of the invention is to reduce the exposure of people to the allergens by providing a toxicologically reliable composition which acts against most airborne allergens such as mite, pollen, pet (feline, canine, etc.), mold and fungus allergens using the synergistic effect of tannic acid with different components and denatures these allergen proteins, and to prevent the occurrence of asthma-allergy symptoms associated thereto.

The most important advantage of the invention is that it eliminates the tertiary structure of the peptide chain by breaking the peptide and hydrogen bonds in amino acids and the salt bridges between the positive and negative side chains. Thus, the physical structure of the allergen protein changes, and the allergen protein is irreversibly denatured.

An advantage of the invention is that the protein exposed to the composition, the physical structure of which is changed, cannot bind to the receptors (Immunoglobin E receptors) found on mast cells in the respiratory tract. This is important. This is because the body does not release histamine as the allergens cannot bind to immunoglobulin E, which causes allergy and asthma attacks, thereby providing a relief and recovery in patients with allergy depending on reduction or cessation of histamine release.

Another advantage of the invention is that it denatures the allergen proteins while they are still in the environment and prevents the exposure of sensitive people to these allergens. Thus, the use of drugs by people, such as decongestants, antihistamines, and corticosteroids is effectively reduced, preventing them from being exposed to the side effects of these drugs.

Another advantage of the invention is that it eliminates processes requiring effort and time, such as cleaning with a cleaning cloth or vacuum cleaner, dusting, the storage of certain textile products in deep freezers like mattress cover-pillow case etc., in order to remove the allergens from the environment.

Another advantage of the invention is the high efficiency thereof. The composition of the invention is effective against most airborne allergens. In the state of the art, the existing products affect only a specific group, while the composition of the invention affects many allergen groups, especially pollen, pet, mite and mold-fungus allergens.

A further advantage of the invention is that it denatures the allergens by manually spraying by means of a trigger bottle or, if desired, by placing it into an electric air freshener device and spraying automatically, or by placing the composition in a water reservoir of a device by means of a steam machine. The existing systems only denature the allergens on the floor surface, while, with the composition of the invention, many different textile surfaces such as sofas, armchairs, beds and curtains, which have allergen-retaining properties, are covered with the composition by spraying, thereby expanding the area of effect of the product and reducing the exposure to allergens to a great extent. In other words, a product which has an increased easy of use and an expanded area of effect is provided with the composition of the invention.

Another advantage of the invention is that it does not contain alcohol and is water-based. Thus, the flammability and/or glowing, which may cause safety problems, is eliminated. In addition, the toxicological reliability of the product is superior to the state of the art, as the product does not contain any biocidal and harmful chemicals.

In order for the subject of the invention to be better understood, the reference will be made to the following drawings.

### DESCRIPTION OF THE FIGURES

FIG. 1 is a view of the molecular form of cyclodextrin in the state of the art.

FIG. 2 is a schematic view of the structure levels of the protein in the state of the art.

FIG. 3 is a schematic view of the reversible denaturation mechanism of the allergen protein during the denaturation of the allergens with the second version of the composition of the invention.

FIG. 4 is a schematic view of the binding mechanism of the allergenic substance to a mast cell in the state of the art.

### REFERENCE NUMERALS OF THE SECTIONS, PARTS AND FLOW FOR DESCRIBING THE INVENTION

1—Cyclodextrin molecule
   1a—Outer surface
   1b—Inner surface
   1c—Cavity
2—Protein structure levels
   2a—Primary structure level
   2b—Secondary structure level
   2c—Tertiary structure level
   2d—Quaternary structure level
3—Denaturation mechanism
   3a—Protein of tertiary structure
   3b—Protein of primary structure
4—Histamine releasing mechanism from mast cells
   4a—Mast cell
   4b—Histamine
   4c—Receptor
   4d—Immunoglobulin E antibody
   4e—Allergenic substance

### DETAILED DESCRIPTION OF THE INVENTION

The composition of the invention aims at developing a product comprising a water based drug product of vegetable origin, which denatures the mite, pollen, pet (feline, canine, etc.), mold and fungal allergens, thereby reducing the exposure of people to these allergens and preventing the asthma-allergy symptoms associated thereto. In line with this main purpose, two different versions of compositions containing an anionic surfactant or cyclodextrin, together with various acids, herbal essential oils and water have been developed. The invention includes compositions for the denaturation of respiratory allergens as well as the method of administration of said compositions. The term denaturation refers to the destruction and deactivation of the allergens.

The first version of the composition of the invention most generally contains at least one acid, at least one anionic surfactant, at least one herbal essential oil and water. In a preferred embodiment, tannic acid is used as an acid, sodium dodecyl sulfate is used as an anionic surfactant, and clove oil is used as herbal essential oil. Before indicating the ratios of said components, explanations will be made about each component.

In the composition of the invention, at least one of tannic acid, acetic acid, malic acid, citric acid, oleic acid, formic acid, hydrochloric acid, sulfuric acid, lactic acid, tartaric acid, myristic acid, palmitic acid, butyric acid, linoleic acid can be selected as an acid. The preferred embodiment of the invention contains tannic acid. Tannins are polyphenolic compounds generally found in the roots, wood, bark, leaves and fruits of the plants. In the composition of the invention, tannic acid may be obtained from different sources. However, tannic acid of vegetable origin, obtained from tannins in tea, performs chemical denaturation in its natural state. Therefore, tannic acid obtained from tannins in tea is especially preferred. Due to its high affinity for peptide bonds, tannic acid breaks hydrogen bonds and salt bridges between the positive-negative side chains, thereby destructing the tertiary structure of the peptide chain. Tannic acid affects the secondary and tertiary structure of the allergen protein without affecting the primary structure thereof.

In the composition of the present invention, anionic surfactants also affect the primary structure of the allergen protein, causing the protein to be irreversibly denatured and precipitated. At least one of linear alkyl benzene sulfonate (LAS), alcohol ether sulfate (AES), secondary alkane sulfonate (SAS), alcohol sulfate (AS), sodium dodecyl sulfate (SDS), sodium lauryl sulfate (SLS), aluminum lauryl sulfate (ALS), ammonium lauryl ether sulfate (ALES) substances may be used as said anionic surfactants. In a preferred embodiment, sodium dodecyl sulfate (SDS) is used as the anionic surfactant. This is because that, as a result of the experimental studies, it has been observed that tannic acid and sodium dodecyl sulfate (SDS) form an excellent synergy in increasing the capacity of allergen denaturation. The excellent synergy mentioned here is that the composition has a strong effect against allergens, showing high efficacy even when applied at low concentrations.

In the composition of the invention, at least one of clove oil, rosemary oil, thyme oil, olive oil, peppermint oil, eucalyptus oil, lavender oil, tea tree oil, thus, bergamot oil, lemon oil, orange oil, sea buckthorn oil, cedar wood oil, sandalwood oil, and tangerine oil may be used as the herbal essential oil. However, clove oil is especially preferred due to the eugenol compound contained therein. This is because eugenol, the antimicrobial and antiviral effects of which are known in the state of the art, is also used as a biocide. Accordingly, the clove oil denatures allergens due to the polyphenol content thereof in the composition of the invention, while it also inactivates organisms such as mites. In other words, the polyphenol in the herbal essential oils in the composition of the invention contributes to the irreversible denaturation of the allergens by showing a synergistic effect with the acid, thus increasing the protein denaturation strength.

The ratios of said components in the composition of the invention are given in the table below. The lower and upper limit values determined here are the value ranges determined as a result of long experimental efforts.

TABLE 1
Composition - Version 1 for the Denaturation
of the Respiratory Allergens

| | Percentage by | Preferred Percentage |
|---|---|---|
| Component | Weight (%) | by Weight (%) |
| Acid | 0.1-5 | 0.1-0.4 |
| (Preferably Tannic Acid) | | |
| Anionic Surfactant | 0.1-5 | 0.1-0.4 |
| (Preferably Sodium Dodecyl | | |
| Sulfate (SDS)) | | |
| Herbal Essential Oil | 0.1-5 | 0.1-0.4 |
| (Preferably Clove Oil) | | |
| Water | In the remaining | Water in the remaining |
| | amount | amount |

As provided in Table 1, the composition of the invention comprises, by weight, 0.1-5%, preferably 0.1-0.4% of an acid, 0.1-5%, preferably 0.1-0.4% of an anionic surfactant and 0.1-5%, preferably 0.1-0.4% of herbal essential oil, and the remaining is water. Here, the term “water in the remaining amount” means that it will brought to a total of 100 with water if the other components are selected within the specified limits. For example, when 0.5% of acid, 3% of anionic surfactant, 0.5% of herbal essential oil are selected, the amount of water should be 96%. The ratios given here are by weight.

In the composition of the invention, the acid is at least one of tannic acid, acetic acid, malic acid, citric acid, oleic acid, formic acid, hydrochloric acid, sulfuric acid, lactic acid, tartaric acid, myristic acid, palmitic acid, butyric acid, linoleic acid. The preferred embodiment of the invention contains tannic acid.

The anionic surfactant in said content is at least one of linear alkyl benzene sulfonate (LAS), alcohol ether sulfate (AES), secondary alkane sulfonate (SAS), alcohol sulfate (AS), sodium dodecyl sulfate (SDS), sodium lauryl sulfate (SLS), aluminum lauryl sulfate (ALS), ammonium lauryl ether sulfate (ALES) substances. In a preferred embodiment, sodium dodecyl sulfate (SDS) is used as the anionic surfactant.

The herbal essential oil in said content is at least one of clove oil, rosemary oil, thyme oil, olive oil, peppermint oil, eucalyptus oil, lavender oil, tea tree oil, thus, bergamot oil, lemon oil, orange oil, sea buckthorn oil, cedar wood oil, sandalwood oil, and tangerine oil. In a preferred embodiment, the clove oil (eugenol) is used as the herbal essential oil.

In a second version of the invention, a composition has been developed, which irreversibly denaturates the allergens passing through the airway in a chemical way in the environment, by forming an inclusion complex between cyclodextrin and the acid and providing a synergistic effect with the polyphenol content in the herbal essential oil. Accordingly, the composition of the invention generally contains at least one acid, at least one cyclodextrin, at least one herbal essential oil and water. Before indicating the ratios of said components, explanations will be made about each component and the technical effects thereof.

Cyclodextrins (CD) are molecular cages produced industrially as a result of the degradation of starch by the transglycosylase enzyme produced by many microorganisms (such as Bacillus). FIG. 1 shows the molecular form of cyclodextrin. Accordingly, the cyclodextrin molecule 1 has a polar hydrophilic outer surface 1a and a hydrophobic inner surface 1b which enables them to host hydrophobic compounds in a hydrophilic medium. The complex formed by filling the cavity inside the cyclodextrin molecule 1 by another molecule is called an inclusion complex. In the composition of the invention, the hydrophobic inner surface 1b of the cyclodextrin molecule 1 captures allergen proteins. Said capturing mechanism occurs due to the hydrophobic attraction forces formed between the allergen proteins of a hydrophobic structure and the inner surface 1b of a hydrophobic structure. In the composition of the invention, cyclodextrin may be selected from at least one of natural cyclodextrins, methylated cyclodextrins, hydroxypropyl cyclodextrins, sulfobutylether-β-cyclodextrins, branched cyclodextrins, acylated cyclodextrins, ionized cyclodextrins, carboxymethylethyl-β-cyclodextrins, sulphated cyclodextrins. Preferably, beta cyclodextrin, which is one of the natural cyclodextrin derivatives, is used.

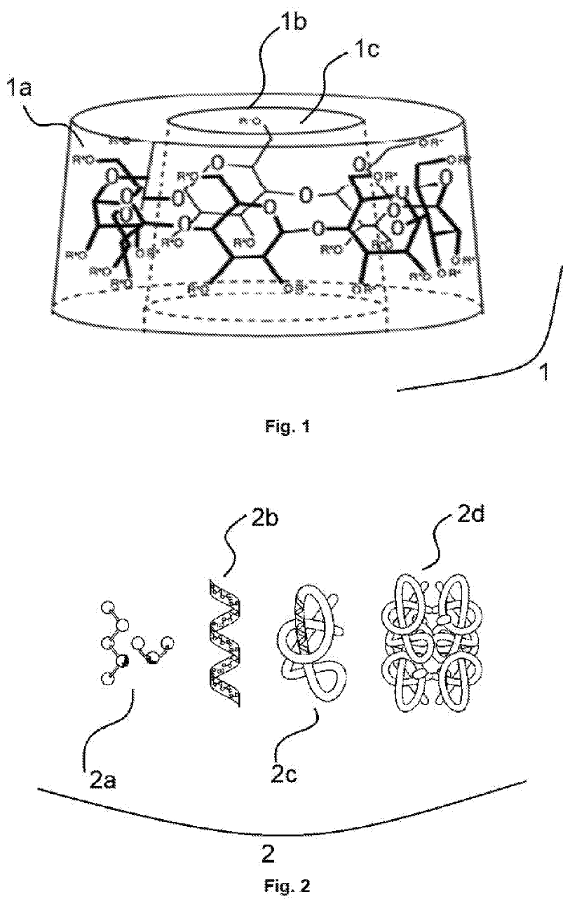

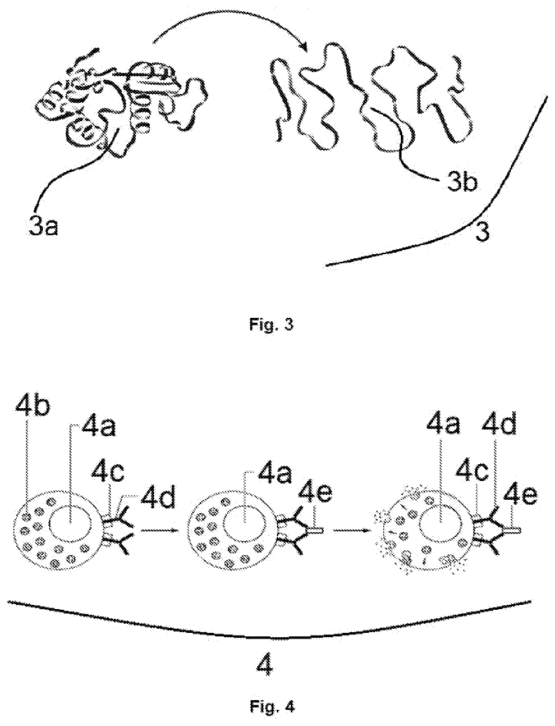

In the composition of the invention, an inclusion complex is formed, using cyclodextrin and an acid. An inclusion complex was formed by filling the cavity 1c inside the cyclodextrin molecule 1 shown in FIG. 1 with the acid molecules. The acid mentioned above is preferably tannic acid, especially tannic acid derived from tannins in tea. Tannic acid of vegetable origin, derived from the tannins in tea, chemically denaturates the protein of natural state. Under normal conditions, there are different levels of protein structure. In the state of the art, the levels of protein structure 2 are shown in FIG. 2. A linear polymer (polypeptide) chain formed by amino acids linked to each other by peptide bonds represents the primary structure level of the protein 2a. The secondary structure level of the protein 2b is defined by the repeating coils and folds, which affect the overall conformation thereof. These folds are formed by hydrogen bonds formed equally spaced along the polypeptide backbone. The tertiary structure level 2c formed by the folding of the secondary structural elements at a higher level results especially from the interactions between the R groups (a Radical, a variable side chain). The main interactions involved in the three-dimensional formation with the tertiary structure level 2c are hydrophobic and Van der Waals interactions. In addition, ionic bonds, salt bridges and hydrogen bonds also play a role in the formation of this structure level. In some proteins, the functional form is obtained by joining the subunits of two or more polypeptide chains. A quaternary structure level 2d is mentioned here. Proteins which have a three-dimensional structure and are biologically active are called natural proteins, which means their structure is intact. Denaturation means deterioration of the three-dimensional structure of a protein without breaking the peptide bonds and the loss of activity. The denaturation mechanism (3) during the denaturation of allergen proteins with the compound of the invention is schematically shown in FIG. 3. Here, there is a mechanism in which the protein of tertiary structure 3a is converted to the protein of primary structure 3b. This step is performed by tannic acid breaking the hydrogen bonds and salt bridges between the positive-negative side chains due its high affinity for peptide bonds. Tannic acid disrupts the physical structure of the molecule by breaking the bonds in the secondary and tertiary structures without affecting the primary structure of allergen proteins. The composition of the invention comprises at least one of tannic acid, acetic acid, malic acid, citric acid, oleic acid, formic acid, hydrochloric acid, sulfuric acid, lactic acid, tartaric acid, myristic acid, palmitic acid, butyric acid, linoleic acid. The use of tannic acid obtained from the tannins in tea leaves is especially preferred.

The denaturation mechanism (3) shown in FIG. 3 may occur reversibly or irreversibly under normal conditions. This means that the protein returning or not returning to its original state after being denatured. In other words, when the protein of the primary structure 3a finds a cavity, it may fold again into a secondary or tertiary form. The polyphenol in the herbal essential oils in the composition of the invention contributes to the irreversible denaturation by showing a synergistic effect with the acid (preferably tannic acid), thus increasing the protein denaturation strength. At least one of clove oil, rosemary oil, thyme oil, olive oil, peppermint oil, eucalyptus oil, lavender oil, tea tree oil, thus, bergamot oil, lemon oil, orange oil, sea buckthorn oil, cedar wood oil, sandalwood oil, and tangerine oil may be used as the herbal essential oil. The composition of the invention preferably comprises rosemary oil.

The ratios of said components in the composition of the invention are given in the table below. The lower and upper limit values determined here are the value ranges determined as a result of long experimental efforts.

TABLE 2
Composition - Version 2 for the Denaturation
of the Respiratory Allergens

| | Percentage by | Preferred Percentage |
|---|---|---|
| Component | Weight (%) | by Weight (%) |
| Acid (Preferably Tannic | 0.01-10 | 0.1-8 |
| Acid) | | |
| Cyclodextrin (CD) | 0.01-5 | 0.1-4 |
| (Preferably Beta | | |
| Cyclodextrin) | | |
| Herbal Essential Oil | 0.01-10 | 0.1-5 |
| (Preferably Rosemary Oil) | | |
| Water | In the remaining | In the remaining |
| | amount | amount |

As provided in Table 2, the composition of the invention comprises, by weight, 0.01-10%, preferably 0.1-8% of an acid, 0.01-5%, preferably 0.1-4% of cyclodextrin and 0.01-10%, preferably 0.1-5% of herbal essential oil, and the remaining is water. Here, the term “water in the remaining amount” as described above means that it will brought to a total of 100 with water if the other components are selected within the specified limits. The ratios given in Table 2 are by weight. Cyclodextrin is preferably beta cyclodextrin. However, if desired, at least one of natural cyclodextrins, methylated cyclodextrins, hydroxypropyl cyclodextrins, sulfobutylether-β-cyclodextrins, branched cyclodextrins, acylated cyclodextrins, ionized cyclodextrins, carboxymethylethyl-β-cyclodextrins, sulphated cyclodextrins may be selected as cyclodextrin. The herbal essential oil is preferably rosemary oil. The herbal essential oil is at least one of clove oil, rosemary oil, thyme oil, olive oil, peppermint oil, eucalyptus oil, lavender oil, tea tree oil, thus, bergamot oil, lemon oil, orange oil, sea buckthorn oil, cedar wood oil, sandalwood oil, and tangerine oil. In a preferred embodiment of the invention, the herbal essential oil is preferably rosemary oil.

The second version of the composition of the invention for denaturating the allergens has an advantage over the first version as follows: in the first version, the composition acts by randomly contacting with the allergen proteins. In the second version, an attraction force occurs due to the hydrophobic interactions between the hydrophobic structure of the inner surface of the cyclodextrin 1b and the hydrophobic structure of the allergen proteins. Due to the effect of this attraction force, the allergen protein is strongly captured and is denaturated by irreversibly degrading the protein structure by the effect of tannic acid and herbal essential oil in the molecular cage. This denaturation of the allergen protein with the composition of the invention benefits the patients with allergy as follows:

The mechanism of histamine release from mast cells 4 when an allergic patient is exposed to an allergen in the state of the art is schematically shown in FIG. 4. Mast cells 4a are connective tissue cells with granules rich in heparin and histamine 4b. It is usually located in every part of the body, but it is especially found in areas which are in contact with the external factors. Therefore, it is encountered in abundance in the mucosa and is responsible for protecting the body from toxins and various organisms. When the allergenic substance 4e in the environment enters the body through the respiratory tract, it is detected by a receptor 4c. Thereupon, the allergenic substance 4e is captured by the immunoglobulin E antibody 4d in the mast cells 4e, and histamine 4b is released from the mast cells 4a. As seen in FIG. 4, there is a mechanical lock-key relationship during the binding of the allergenic substance 4e to the immunoglobulin E antibody 4d, as in the enzyme-substrate interaction. Accordingly, the allergens are denatured even before they enter the body through the respiratory tract by the composition of the invention, thus the binding of the allergenic substance 4e to the immunoglobulin E antibody 4d is prevented and the histamine 4b release of the body is ceased, thus preventing the allergic reactions.

The compound of the invention has a superiority for two aspects when evaluated for the mechanism of histamine release from mast cells (4), that is, the allergic reaction:

Firstly, as mentioned above, as a result of the hydrophobic attraction forces between the hydrophobic inner surface of the cyclodextrin 1b and the hydrophobic allergen protein, the allergen protein is strongly captured by being received into the cavity 1c, and the allergen protein is irreversibly denatured by the effect of tannic acid and herbal essential oil in the composition. As the allergen protein denatured will be in the primary structure, there will be no lock-key compatibility with the immunoglobulin E antibody, and the allergenic substance 4e will not be able to bind to the immunoglobulin E antibody 4d. This means that histamine 4b is not released from mast cells 4a and allergic reactions do not occur.

In a second scenario, if the allergenic substance 4e received into the body by inhalation cannot be denatured as expected (i.e., if the protein structure cannot be converted to the primary structure), it will again become very difficult for the allergenic substance 4e to bind to the immunoglobulin E antibody 4d thanks to the composition of the invention. This is because: In the mechanism of histamine release from mast cells 4 shown in FIG. 4, the binding of the allergenic substance 4e detected by the receptor 4c to the immunoglobulin E antibody 4d is due to the effect of hydrophobic attraction force. As a result of the hydrophobic interaction between the cyclodextrin and the allergen protein in the composition of the invention, the allergenic substance 4e becomes neutral. Therefore, when it reaches the mast cells (4a) from the nasal cavity through respiration, the expected hydrophobic attraction will not occur with the immunoglobulin E antibody 4d, as the hydrophobic structure thereof is disrupted. As the binding of the allergenic substance 4e to the immunoglobulin E antibody 4d becomes difficult, the occurrence of allergic reactions will be slowed and mitigated.

The compositions of the invention are effective against the respiratory allergens such as mite, pollen, feline, canine, mold and fungal allergens. Said respiratory allergens may be DER P1, DER P2 ve DER F1, DER F2 mite allergens in the state of the art; Fel d 1, Fel d 2 and Can f 1, Can f 2 pet allergens; rAlt a 1, rAsp f1, rCla h 8, Aspergillus fumigatus, Penicilium notatum mold and fungal allergens; and Betula pendula (Common birch), Quercus L. (Oak tree), Arnica chamissonis (Grass), Populus (Poplar), Salix Salicaceae pollen allergens.

The invention also includes administration methods of the above-mentioned compositions for the denaturation of respiratory allergens. The application method of the composition of the invention comprises the following processes:
spraying the composition manually via a trigger spray bottle,
spraying the composition automatically by an electric air freshener device upon placement therein
and/or
denaturating the allergens by placing the composition in the water reservoir of a steam machine.

### INDUSTRIAL APPLICABILITY OF THE INVENTION

The compositions of the invention are in the form of aqueous solutions. It denatures the allergens by being manually sprayed by means of a trigger spray bottle or, if desired, by placing it into an electric air freshener device and spraying automatically, or by placing the composition in a water reservoir of a device by means of a steam machine. The compounds of the present invention show high activity against most respiratory allergens such as mite, pollen, pet (feline, canine, etc.), mold and fungus allergens. The denaturation of the allergen substances occur in an external environment to which the product is applied, but not in the body.

1. A composition for the denaturation of the respiratory allergens, comprising, by weight, 0.01-10%, preferably 0.1-8% of an acid, 0.01-5%, preferably 0.1-4% of cyclodextrin and 0.01-10%, preferably 0.1-5% of herbal essential oil, and the remaining is water.

2. A composition according to claim 1, wherein said acid is at least one of tannic acid, acetic acid, malic acid, citric acid, oleic acid, formic acid, hydrochloric acid, sulfuric acid, lactic acid, tartaric acid, myristic acid, palmitic acid, butyric acid, linoleic acid.

3. A composition according to claim 1, wherein said acid is tannic acid.

4. A composition according to claim 3, wherein said tannic acid is obtained from tannins in the tea leaves.

5. A composition according to claim 1, wherein said cyclodextrin is at least one of natural cyclodextrins, methylated cyclodextrins, hydroxypropyl cyclodextrins, sulfobutylether-β-cyclodextrins, branched cyclodextrins, acylated cyclodextrins, ionized cyclodextrins, carboxymethylethyl-β-cyclodextrins, sulphated cyclodextrins.

6. A composition according to claim 1, wherein said cyclodextrin is beta cyclodextrin.

7. A composition according to claim 1, wherein said herbal essential oil is at least one of rosemary oil, clove oil, thyme oil, olive oil, peppermint oil, eucalyptus oil, lavender oil, tea tree oil, thus, bergamot oil, lemon oil, orange oil, sea buckthorn oil, cedar wood oil, sandalwood oil, and tangerine oil.

8. A composition according to claim 1, wherein said herbal essential oil is rosemary oil.

9. An alternative composition for the denaturation of the respiratory allergens, comprising, by weight, 0.1-5%, preferably 0.1-0.4% of an acid, 0.1-5%, preferably 0.1-0.4% of an anionic surfactant and 0.1-5%, preferably 0.1-0.4% of herbal essential oil, and the remaining is water.

10. A composition according to claim 9, wherein said acid is at least one of tannic acid, acetic acid, malic acid, citric acid, oleic acid, formic acid, hydrochloric acid, sulfuric acid, lactic acid, tartaric acid, myristic acid, palmitic acid, butyric acid, linoleic acid.

11. A composition according to claim 9, wherein said acid is tannic acid.

12. A composition according to claim 11, wherein said tannic acid is obtained from tannins in the tea leaves.

13. A composition according to claim 9, wherein said anionic surfactant is at least one of linear alkyl benzene sulfonate (LAS), alcohol ether sulfate (AES), secondary alkane sulfonate (SAS), alcohol sulfate (AS), sodium dodecyl sulfate (SDS), sodium lauryl sulfate (SLS), aluminum lauryl sulfate (ALS), ammonium lauryl ether sulfate (ALES) substances.

14. A composition according to claim 9, wherein said anionic surfactant is preferably sodium dodecyl sulfate (SDS).

15. A composition according to claim 9, wherein said herbal essential oil is at least one of clove oil, rosemary oil, thyme oil, olive oil, peppermint oil, eucalyptus oil, lavender oil, tea tree oil, thus, bergamot oil, lemon oil, orange oil, sea buckthorn oil, cedar wood oil, sandalwood oil, and tangerine oil.

16. A composition according to claim 9, wherein said herbal essential oil is clove oil (eugenol).

17. A composition according to claim 9, wherein the allergens for which said composition is effective are mite, pollen, feline, canine, mold and fungal allergens.

18. A composition according to claim 17, wherein said allergens are DER P1, DER P2 ve DER F1, DER F2 mite allergens; Fel d 1, Fel d 2 and Can f1, Can f2 pet allergens; rAlt a 1, rAsp f1, rCla h 8, Aspergillus fumigatus, Penicillium notatum mold and fungal allergens; and Betula pendula (Common birch), Quercus L. (Oak tree), Arnica chamissonis (Grass), Populus (Poplar), Salix Salicaceae pollen allergens.

19. A method of application of the composition for the denaturation of respiratory allergens according to claim 1, comprising the following process steps:
spraying the composition manually via a trigger spray bottle;
spraying the composition automatically by an electric air freshener device upon placement therein; and/or
denaturating the allergens by placing the composition in the water reservoir of a steam machine.
